(19) United States
(12) Patent Application Publication
(10) Pub. No.: US 2011/0307216 A1
(43) Pub. Date: Dec. 15, 2011

(54) METHOD FOR AUTOMATED MEASUREMENT OF EYE-TRACKING SYSTEM RANDOM ERROR

(75) Inventor: George H. Lindquist, Ann Arbor, MI (US)

(73) Assignee: OptiMetrics, Inc., Ann Arbor, MI (US)

(21) Appl. No.: 13/157,964

(22) Filed: Jun. 10, 2011

Related U.S. Application Data
(60) Provisional application No. 61/353,349, filed on Jun. 10, 2010.

(51) Int. Cl. G06F 15/00; A61B 3/113

(52) U.S. Cl. 702/182

(57) ABSTRACT

An estimate of random error in an eye-tracking system is done directly from eye-tracker outputs during a trial, without the need for an explicit calibration process. The distances traveled between adjacent user observations are computed, and the random error δᵣ of the eye-tracker system is estimated using the statistical distribution of the computed distances. In the preferred embodiment, the distances traveled between adjacent observations is sampled on a continuous basis. The process includes measuring the mode (peak value) of the distribution in observation distances. These values are sorted by increasing distance, and a window of about 50 observations is used to estimate the mode of the distance distribution. A running estimate of the mode is computed, and the result is divided by a constant. A preferred constant of 1.61 was derived using a series of Monte Carlo simulations.

### REFERENCE TO RELATED APPLICATION

This application claims priority from U.S. Provisional Patent Application Ser. No. 61/353,349, filed Jun. 10, 2010, the entire content of which is incorporated herein by reference.

### FIELD OF THE INVENTION

This invention is a method to automatically estimate the random error in an eye-tracking system in normal operation, without requiring an explicit calibration period or process. It is less intrusive and more accurate than an explicit calibration and also supports the broader use of eye-tracking systems in education and training.

### BACKGROUND OF THE INVENTION

Eye-tracking systems are powerful tools used for research in human cognition and for practical applications in understanding human perception. They are useful in designing effective advertising and human-machine interfaces. Despite the recognized value of these systems, widespread application of the technology has been limited by difficulties in association of the output data with specific scene/screen objects and by the high cost of the devices. These problems are related; high accuracy devices are needed to support correct object association, and such devices are expensive.

A variety of “random” and systematic errors affect the accuracy of eye-trackers. Random errors result from factors such as electronic noise, angular resolution limits of the sensor, and mechanical vibrations. Random errors can also be introduced by human physiological variations such as variations in the shape of the eye(s), variations in eye(s) resolution, and variations in fixation stability.

Analysis of eye-tracking system outputs requires the separation (or segmentation) of periods when the eye's focus region (foveal region) is moving (saccade) and when the foveal region is stationary (fixation). It is during a fixation when a subject is “looking” at a specific scene object. In the analysis of eye-tracking data, it is critical to separate the saccade periods from the fixation periods. During saccades, the eye view moves quickly from region to region. During fixations, the eye is not quite stationary; it exhibits “micro-movements” around the fixation point. The output from an eye-tracking system is normally processed with an automated algorithm that attempts to separate (segment) fixation periods from saccade periods. Accurate separation of fixation periods is critical to associating a fixation with a specific scene object.

Segmentation algorithms rely on analysis of the speed and distance of a candidate eye movement to classify that movement as being a part of a saccade or a fixation period. The knowledge of the random noise level in the eye-tracking system is a critical parameter necessary for accurate segmentation. An automated method for estimating the random noise level in the eye-tracking system is the subject of this disclosure.

The error in an eye-tracking system is the difference between the true focused (foveal) angular direction and the foveal direction reported by the eye-tracking system. The total error can be divided into two components, a random component and a systematic component.

The random component results from random processes in the electronic and optical components of the eye-tracking system and random components introduced by the human subject. Humans vary in their abilities to move and stabilize their sight lines. Those human factors vary with visual acuity and are also affected by alertness and disease. Because they are random, these error components cannot be corrected by calibration. This type of error can be represented by a standard deviation, namely δᵣ.

The systematic component results from such factors as a pose shift, the shift of the eye-tracking optic on the subject's head, and a change in shape of the eye surface with temperature and/or moisture. To the extent these factors are constant, or slowly varying with time, the resulting systematic error can be accounted for and eliminated using a simple calibration process.

In an eye-tracker calibration process, the human subject is asked to look at each of a series of points on a screen for a few seconds. The known geometry of the points and the subject's eye position is then used to calculate the true viewing angles. Those angles are then compared with the viewing angles reported by the eye-tracking system, and the differences are computed to determine the error(s) in the system. The mean differences (offsets) between the true viewing angles and the reported viewing angles are recorded and can be used to calibrate (correct) the output data for systematic errors. Once the mean offsets are applied as corrections to the measured viewing angles, the random error δᵣ can be calculated.

While this calibration process is effective in determination of the random error δᵣ, it must be repeated for each subject and generally before and after each trial. This requirement adds to the complexity and cost of using eye-tracking systems in routine applications. It is also possible that the random error may vary during a trial; such a variation may compromise the quality of the data. A method to determine the δᵣ continuously throughout a trial without an explicit calibration process would improve the ease of use and the quality of eye-tracking data.

### SUMMARY OF THE INVENTION

This invention is directed to estimating random error in an eye-tracking system. In the preferred embodiment, this determination is done directly from eye-tracker outputs during a trial, without the need for an explicit calibration process. The method is practiced in conjunction with an eye-tracking system operative to determine the position of a user's eye(s) in conjunction with observations made by the user. The invention is not limited in terms of the type of eye tracker system used.

In accordance with the method, the distances traveled between adjacent user observations are computed, and the random error δᵣ of the eye-tracker system is estimated using the statistical distribution of the computed distances. In the preferred embodiment, the distances traveled between adjacent observations is sampled on a continuous basis over multiple observations over 100 or more. The distances between adjacent observations may be sampled at a rate of 60 samples per second, more or less.

The process includes the step of measuring the mode (peak value) of the distribution in observation distances. These values are sorted by increasing distance, and a window of about 50 observations is used to estimate the mode of the distance distribution. A running estimate of the mode is computed, and the result is divided by a constant. A preferred constant of 1.61 was derived using a series of Monte Carlo simulations. Thus, according to the invention δᵣ may be defined as the most likely distance between adjacent observations divided by 1.61.

Once δᵣ has been determined it can be used for various purposes, including separation of saccade periods from fixation periods in segmentation algorithms. The result may also be used to estimate the utility of a specific eye-tracking trial or monitor the overall “health” of the eye-tracker system.

### DETAILED DESCRIPTION OF THE INVENTION

This invention resides in a method of determining random error, δᵣ, directly from eye-tracker outputs during a trial, without the need for an explicit calibration process. The invention is not limited in terms of the eye-tracking system or technology used to observe eye movements. Commonly assigned U.S. Pat. No. 7,872,635, the entire content of which is incorporated herein by reference, describes examples of eye-tracking systems to which this invention is applicable.

In deriving the method, it has been observed that fixations take up at least as much time as saccades for typical visual tasks. Furthermore, in a saccade, the eye travels over large distances, while during a fixation, the eye motion is small and dominated by random errors. In accordance with the instant invention, we use the statistics of the observed distance traveled between adjacent observations of eye position to estimate the random error δᵣ.

The statistics of the distance traveled between sequential eye position observations during saccades are determined by the trajectories taken during saccades. During saccades, large-scale eye motion occurs. The distances traveled between adjacent observations are likely to be large and diverse, depending on the saccade trajectory and the timing of the observations. During fixations, however, the eye motion is small and the distances traveled between adjacent observations are determined primarily by the random error in the observed position. Thus the statistics of this distance traveled between adjacent observations differ between saccades and fixations.

During saccades, both the mean and the spread of this distribution are expected to be large, leading to a wide, flat distribution. In contrast, during fixations the distances are expected to be small, with both mean and spread determined by the random error of the eye-tracker. Carpenter¹ observed that time is approximately evenly split between fixations and saccades for a human subject performing a typical visual task. As a result, the combined distribution of distance traveled will appear as roughly the sum of these two distributions, one wide and flat, the other narrower and more strongly peaked.

Because of this difference in the two components, one would expect that the mode (the peak value) of the combined distribution will likely be dominated by the peak of the highest, most concentrated component. Thus the mode of the distribution of distance traveled between adjacent observations is likely to be a good indicator of the mode of the distance traveled during fixations, which, in turn is primarily determined by the random error of the observation process.

Thus, in accordance with the method aspect of the invention, we continually sample the distance traveled between adjacent observations, and buffer the values obtained, while concurrently and continually measuring the mode (peak value) of its distribution. We use the procedure outlined in Press et al.² to compute a running estimate of this mode. From our own simulations, this value, divided by 1.61, is a reliable measure of the one-dimensional random error of the eye-tracker process.

More particularly, we continually record the eye-tracker output, calculating values for the distance between adjacent observations over several hundred observations. A sampling rate of 60 hertz is sufficient for nominal experimental scenarios. We then sort these distance values by increasing distance, and use a window of about 50 observations to estimate the mode of the distance distribution. This most likely distance value is approximately 1.61δᵣ, as determined by a series of Monte Carlo simulations. Thus, we estimate δᵣ as: the most likely distance between adjacent looks divided by 1.61.

Once δᵣ has been determined it can be used to:

1. separate saccade periods from fixation periods in the segmentation algorithms.

2. estimate the utility of a specific trial. If δᵣ is too large, the trial results may contain too much “noise” to be useful.

3. monitor the overall “health” of the eye-tracker system. A large error may be the result of a hardware system problem or a human subject that is unsuitable for an eye-tracking experiment.

1. A method of determining random error in an eye-tracking system, comprising the steps of:
providing an eye-tracking system operative to determine the position of a user's eye in conjunction with observations made by the user;
computing the distances traveled between adjacent user observations; and
estimating the random error δᵣ of the eye-tracker system using the statistical distribution of the computed distances.

2. The method of claim 1, including the step of continuously sampling the distance traveled between adjacent observations.

3. The method of claim 1, including the step of sampling the distance between adjacent observations over at least 100 observations.

4. The method of claim 1, including the step of sampling the distance between adjacent observations over at a rate of about 60 samples per second.

5. The method of claim 1, including the step of measuring the mode (peak value) of the distribution.

6. The method of claim 1, including the steps of:
sorting the distance values by increasing distance, and
using a window of about 50 observations to estimate the mode of the distance distribution.

7. The method of claim 1, including the steps of:
measuring the mode (peak value) of the distribution; and
computing a running estimate of the mode.

8. The method of claim 1, including the steps of:
measuring the mode (peak value) of the distribution;
computing a running estimate of the mode; and
dividing the running estimate by a constant.

9. The method of claim 1, including the steps of:
measuring the mode (peak value) of the distribution;
computing a running estimate of the mode;
deriving a constant using a series of Monte Carlo simulations; and
dividing the running estimate by the constant.

10. The method of claim 1, including the steps of:
measuring the mode (peak value) of the distribution;
computing a running estimate of the mode; and
dividing the running estimate by 1.61.

11. The method of claim 1, wherein δᵣ is defined as the most likely distance between adjacent observations divided by 1.61.

12. The method of claim 1, including the step of using the estimate of δᵣ to segment saccade periods from fixation periods.

13. The method of claim 1, including the step of using the estimate of δᵣ to determine whether an eye-tracking session has too much noise to be useful.

14. The method of claim 1, including the step of using the estimate of δᵣ to determine whether the eye-tracking system is functioning properly or whether the user is unsuitable for an eye-tracking experiment.
